(19) United States
(12) Patent Application Publication
(10) Pub. No.: US 2026/0306514 A1
(43) Pub. Date: Oct. 8, 2026

(54) ORAL PHARMACEUTICAL FORMULATIONS CONTAINING 4-((2-HYDROXY-3-METHOXYBENZYL)AMINO) BENZENESULFONAMIDE DERIVATIVES

(71) Applicant: Cadrenal Therapeutics, Inc., Ponte Vedra, FL (US)

(72) Inventors: David Maloney, Point of Rocks, MD (US); Henry Flanner, Montgomery Village, MD (US); Matthew Boxer, New Market, MD (US)

(21) Appl. No.: 19/125,889

(22) PCT Filed: Nov. 2, 2023
(86) PCT No.: PCT/US2023/078471

§ 371 Date: Apr. 30, 2025

Related U.S. Application Data
(60) Provisional application No. 63/382,032, filed on Nov. 2, 2022.

(51) Int. Cl. A61K 31/635; A61K 9/00; A61K 9/16; A61K 31/63

(52) U.S. Cl. CPC A61K 31/635; A61K 9/0053; A61K 9/1617; A61K 9/1635; A61K 31/63

(57) ABSTRACT

Provided are pharmaceutical formulations comprising 4-((2-hydroxy-3-methoxybenzyl)amino)-benzenesulfonamide 12-LOX inhibitors with improved solubility for oral administration. Also provides are pharmaceutical formulations comprising selective 12-LOX inhibitors with high drug loading of 30% or more amorphously dispersed in selected from vinylpyrollidone-vinyl acetate copolymers (Kollidon VA64), amino methacrylate copolymer (Eudragit EPO), Hydroxypropylmethylcellulose E3 (HPMC E3), or a combination thereof and make up 40 to 80% of the total solid content of the formulation. The formulation alternatively contains additives such as surfactants (sodium lauryl sulfate), crystal inhibitors, or pH modifiers up to 20% of the total solid content of the formulation.

### CROSS-REFERENCE TO RELATED PATENT APPLICATIONS

This International Patent Application claims priority to U.S. Provisional Patent Application No. 63/382,032, filed on 2 Nov. 2022, the disclosure of which is incorporated herein in its entirety.

### BACKGROUND OF THE INVENTION

Lipoxygenases are a class of non-heme iron-containing enzymes which regio- and stereospecifically oxidize polyunsaturated fatty acid substrates such as arachidonic acid (AA) and linoleic acid (LA). (Solomon, et al. Chem. Biol. 1997, 4, 795-808; Brash, J. Biol. Chem. 1999, 274, 23679-23682.) The position at which these cis, cis-1,4-pentadiene substrates are oxidized correspond to the requisite lipoxygenase, with the three major human lipoxygenases: 5-LOX, 12-LOX, and 15-LOX-1, oxidizing the C-5, C-12 and C-15 positions respectively. Lipoxygenases are involved in the first committed step in a cascade of metabolic pathways and the products of these enzymes (eicosanoids) are precursors of hormones such as leukotrienes and lipoxins, which mediate a wide array of cellular functions. (Serhan, et al. Chem. Rev. 2011, 111, 5922-5943). Consequently, the lipoxygenase enzymes and their bioactive metabolites (e.g., hydroxyeicosatetraenoic acid (HETE) and leukotriene A4) have been implicated in a variety of inflammatory diseases and cancers.

12-LOX exists as three isozymes, platelet-type, leukocyte, and epidermal, but leukocyte 12-LOX is found in rat, mouse, pig and cow, but not in humans. (Yamamoto Biochim. Biophys. Acta. 1992, 1128, 117-131; Funk et al. FEBS Lett. 1997, 402, 162-166).

12-LOX has been demonstrated to play a role in a number of conditions and/or diseases, such as skin diseases and platelet hemostasis, transplantation/xenotransplantation, cancer (including but not limited to prostate cancer, colorectal cancer, breast cancer and lung cancer), type 1 and type 2 diabetes, diabetic kidney disease (diabetic nephropathy), diabetic nerve disease, diabetic retinopathy, lupus, cardiovascular disease (including but not limited to myocardial infarction, congestive heart failure, heart failure, and stroke), thrombosis, heparin induced thrombocytopenia (HIT), Alzheimer's disease, non-alcoholic steatohepatitis, insulin resistance, and inflammation.

A 4-((2-hydroxy-3-methoxybenzyl)amino)-benzenesulfonamide-based scaffold was identified and used for medicinal chemistry optimization and biological characterization as described in Luci, D K. et al. J. Med. Chem. 2014, 57, 495-506, and U.S. Pat. Nos. 10,266,488 and 10,752,581. This optimization produced several highly potent, selective 12-LOX inhibitors, e.g., N-(benzo[d]thiazol-2-yl)-4-((2-hydroxy-3-methoxybenzyl)amino)benzenesulfonamide (Compound 1), N-(benzo[d]oxazol-2-yl)-4-((2-hydroxy-3-methoxybenzyl)amino)benzenesulfonamide (Compound 2), and 4-((2-hydroxy-3-methoxybenzyl)amino)-N-(3-isopropylphenyl)benzenesulfonamide (Compound 3)

A challenge for the therapeutic use of 4-((2-hydroxy-3-methoxybenzyl)amino)-benzenesulfonamide and related 12-LOX inhibitors are poor aqueous solubility (<5 μM). There exists a need for pharmaceutical formulations of 4-((2-hydroxy-3-methoxybenzyl)amino)-benzenesulfonamide 12-LOX inhibitors.

Relatively insoluble compounds, i.e., solubility in water of less than 200 μg/mL may show promising pharmaceutical activity, but their development as pharmaceuticals, particularly in oral dosage form, present a challenge. Non-aqueous solvents can used in oral pharmaceutical formulations to dissolve water-insoluble drugs. However, these organic solvents, regarded as chemically and biologically inert, may show pharmacological and toxicological effects. These methods are often inadequate for solubilizing enough of a quantity of a drug for an oral formulation. In addition, only therapeutic compounds dissolved in body fluids can permeate biological barriers and reach their site of action on a protein or cell surface or within certain intracellular structures.

### BRIEF SUMMARY OF THE INVENTION

The present application provides pharmaceutical formulations for oral administration, including oral administration, comprising of 4-((2-hydroxy-3-methoxybenzyl)amino)-benzenesulfonamide 12-LOX inhibitors. Preferred are methods of administering these formulations as therapeutic agents in the treatment or prevention of 12-LOX mediated diseases and disorders by oral administration.

In an embodiment, pharmaceutical formulation can comprise:

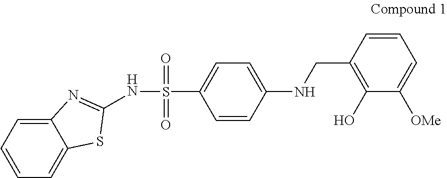 [SMILES: COc1cccc(CNc2ccc(S(=O)(=O)Nc3nc4ccccc4s3)cc2)c1O]

or a pharmaceutically acceptable salt thereof; and a pharmaceutically acceptable carrier.

In an embodiment, pharmaceutical formulation can comprise:

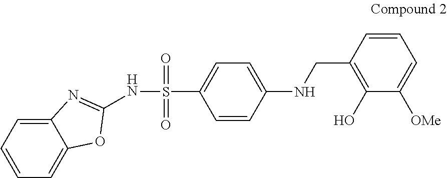 [SMILES: COc1cccc(CNc2ccc(S(=O)(=O)Nc3nc4ccccc4o3)cc2)c1O]

or a pharmaceutically acceptable salt thereof, or diastereomers thereof; and a pharmaceutically acceptable carrier.

In an embodiment, pharmaceutical formulation can comprise:

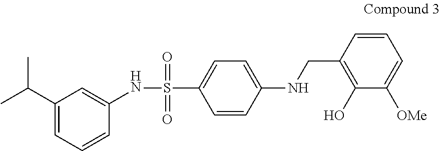 [SMILES: COc1cccc(CNc2ccc(S(=O)(=O)Nc3cccc(C(C)C)c3)cc2)c1O]

or a pharmaceutically acceptable salt thereof, or diastereomers thereof; and a pharmaceutically acceptable carrier.

In an embodiment, pharmaceutical formulation can comprise a compound of Formula (I):

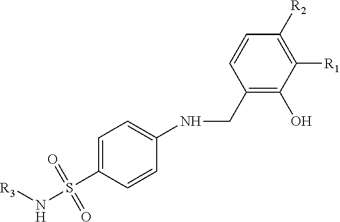

wherein
R₁ is methoxy;
R₂ is H; and
R₃ is selected from the group consisting of 2-benzothiazole, 4-methyl-2-benzothiazole, 6-fluoro-2-benzothiazole, 6-methoxy-2-benzothiazole, 2-benzoxazole, 2-benzimidazole, 2-thiophene, 4-methyl-2-thiazole, 5-methyl-2-thiazole, 4,5-methyl-2-thiazole, 4-phenyl-2-thiazole, 3-quinoline, 8-isoquinoline, phenyl, 1,4-biphenyl, 1-naphthalene, 2-naphthalene, 3-piperazine-phenyl, 4-piperidine-phenyl, 3-piperidine-phenyl, 2-pyridine, 3-pyridine, 4-piperazine-3-pyridine, 3-tert-butyl-phenyl, 3-morpholine-phenyl, 4N-boc-piperidine-3-phenyl, and 3-isopropyl-phenyl, or a pharmaceutically acceptable salt thereof, or diastereomers thereof; and
   a pharmaceutically acceptable carrier.

In an embodiment, pharmaceutical formulation can comprise a pharmaceutical acceptable carrier that is a polymer.

In an embodiment, the pharmaceutical acceptable carrier is a polymer selected from the group consisting of vinylpyrollidone-vinyl acetate copolymers (Kollidon VA64), amino methacrylate copolymer (Eudragit EPO), Hydroxypropylmethylcellulose E3 (HPMC E3), or a combination thereof.

In an embodiment, the pharmaceutical acceptable carrier is a polymer in an amount of between about 40% and 80% of the total solids content. The pharmaceutical acceptable carrier can be a polymer in an amount of between about 40% and 60%, 50% and 80%, 40% and 50%, or 60% and 70% of the total solids content. The pharmaceutical acceptable carrier can be a polymer in an amount of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% of the total solids content.

In an embodiment, the polymer is vinylpyrollidone-vinyl acetate copolymers (Kollidon VA64) and is in an amount of about 40 to 80% of the total solids content.

In an embodiment, the compound of Formula (I) is in an amount between about 1 mg to about 1,000 mg in a pharmaceutical formulation described herein. The compound of Formula (I) can be in an amount between about 1 mg to 100 mg; 250 mg to 500 mg, or 250 mg to 750 mg in a pharmaceutical formulation described herein. The compound of Formula (I) can be in an amount of about 5 mg, 10 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 115 mg, 120 mg, 125 mg, 130 mg, 135 mg. 140 mg, 145 mg, 150 mg, 155 mg, 160 mg, 165 mg. 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg 275 mg, 280 mg, 285 mg, 290 mg, 295 mg, 300 mg, 205 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 625 mg, 655 mg, 675 mg, 700 mg, 725 mg, 750 mg, 775 mg, 800 mg, 825 mg, 850 mg, 875 mg, 900 mg, 925 mg, 950 mg, 975 mg, or 1,000 mg in a pharmaceutical formulation described herein.

In an embodiment, Compound 1 can be in an amount between about 1 mg to about 1,000 mg in a pharmaceutical formulation described herein. Compound 1 can be in an amount between about 1 mg to 100 mg; 250 mg to 500 mg, or 250 mg to 750 mg in a pharmaceutical formulation described herein. Compound 1 can be in an amount of about 5 mg, 10 mg, 20 mg, 25 mg, 30 mg, 35 mg, 40 mg, 45 mg, 50 mg, 55 mg, 60 mg, 65 mg, 70 mg, 75 mg, 80 mg. 85 mg, 90 mg, 95 mg, 100 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg 275 mg, 280 mg, 285 mg, 290 mg, 295 mg, 300 mg, 205 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg. 590 mg, 595 mg, 600 mg, 605 mg, 625 mg, 655 mg, 675 mg, 700 mg, 725 mg, 750 mg, 775 mg, 800 mg, 825 mg, 850 mg, 875 mg, 900 mg, 925 mg, 950 mg, 975 mg, or 1,000 mg in a pharmaceutical formulation described herein.

In an embodiment, Compound 2 can be in an amount between about 1 mg to about 1,000 mg in a pharmaceutical formulation described herein. Compound 2 can be in an amount between about 1 mg to 100 mg; 250 mg to 500 mg, or 250 mg to 750 mg in a pharmaceutical formulation described herein. Compound 2 can be in an amount of about 5 mg, 10 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg 275 mg, 280 mg, 285 mg, 290 mg, 295 mg, 300 mg, 205 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 510 mg, 515 mg, 520 mg, 525 mg, 530 mg, 535 mg. 540 mg, 545 mg, 550 mg, 555 mg, 560 mg, 565 mg, 570 mg, 575 mg, 580 mg, 585 mg, 590 mg, 595 mg, 600 mg, 605 mg, 625 mg, 655 mg, 675 mg, 700 mg, 725 mg, 750 mg, 775 mg, 800 mg, 825 mg, 850 mg, 875 mg, 900 mg, 925 mg, 950 mg, 975 mg, or 1,000 mg in a pharmaceutical formulation described herein.

In an embodiment, Compound 3 can be in an amount between about 1 mg to about 1,000 mg in a pharmaceutical formulation described herein. Compound 3 can be in an amount between about 1 mg to 100 mg; 250 mg to 500 mg, or 250 mg to 750 mg in a pharmaceutical formulation described herein. Compound 3 can be in an amount of about 5 mg, 10 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg. 255 mg, 260 mg, 265 mg, 270 mg 275 mg, 280 mg, 285 mg, 290 mg, 295 mg, 300 mg, 205 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 510 mg, 515 mg, 520 mg, 525 mg, 530 mg, 535 mg, 540 mg. 545 mg, 550 mg, 555 mg, 560 mg, 565 mg, 570 mg, 575 mg, 580 mg, 585 mg, 590 mg, 595 mg, 600 mg, 605 mg, 625 mg, 655 mg, 675 mg, 700 mg, 725 mg, 750 mg, 775 mg, 800 mg, 825 mg, 850 mg, 875 mg, 900 mg, 925 mg, 950 mg, 975 mg, or 1,000 mg in a pharmaceutical formulation described herein.

In an embodiment, the pharmaceutical formulation can further comprise a pharmaceutically acceptable carrier, additive, excipient, preservative, solvent, buffer, or a mixture thereof.

In an embodiment, the pharmaceutical formulation can be suitable for oral administration.

In an embodiment, the pharmaceutical formulation can be formulated for use as a therapeutic agent in the treatment or prevention of 12-LOX mediated diseases and disorders.

In an embodiment, a pharmaceutical composition for treating or preventing a 12-LOX mediated disease and/or disorder can comprise the pharmaceutical formulation described herein.

In an embodiment, a pharmaceutical composition for reducing PAR1-AP or PAR4-AP induced platelet aggregation can comprise the pharmaceutical formulation described herein.

In an embodiment, a pharmaceutical composition for reducing PAR4-AP induced calcium mobilization can comprise the pharmaceutical formulation described herein.

In an embodiment, a pharmaceutical composition for reducing PAR4-AP induced calcium mobilization can comprise the pharmaceutical formulation described herein.

In an embodiment, the pharmaceutical formulation is formulated for oral administration.

In an embodiment, a method for treating or preventing a 12-LOX mediated disease and/or disorder can comprise administering the pharmaceutical formulation described herein to a mammal in need thereof.

In an embodiment, a method for reducing PAR1-AP or PAR4-AP induced platelet aggregation can comprise orally administering to a mammal thereof the pharmaceutical formulation described herein.

In an embodiment, a method for reducing PAR4-AP induced calcium mobilization can comprise orally administering to a mammal thereof the pharmaceutical formulation described herein.

In an embodiment, a method for treating U46619-induced and FcγRIIa-mediated platelet aggregation can comprise orally administering to a mammal thereof the pharmaceutical formulation described herein.

In an embodiment, the mammal is a human.

In an embodiment, the subject in need thereof has a 12-LOX mediated disease and/or disorder.

In an embodiment, the 12-LOX mediated disease and/or disorder is selected from the group consisting of skin diseases and platelet hemostasis, transplantation/xenotransplantation, cancer, optionally prostate cancer, colorectal cancer, breast cancer and lung cancer, type 1 and type 2 diabetes, diabetic nephropathy, optionally diabetic nephropathy, diabetic retinopathy, lupus, cardiovascular disease, optionally myocardial infarction, congestive heart failure, heart failure, and stroke, thrombosis, heparin-induced thrombocytopenia (HIT), Alzheimer's disease, non-alcoholic steatohepatitis, insulin resistance, and inflammation. The 12-LOX mediated disease and/or disorder can be a combination of skin diseases and platelet hemostasis, transplantation/xenotransplantation, cancer, optionally prostate cancer, colorectal cancer, breast cancer and lung cancer, type 1 and type 2 diabetes, diabetic nephropathy, optionally diabetic nephropathy, diabetic retinopathy, lupus, cardiovascular disease, optionally myocardial infarction, congestive heart failure, heart failure, and stroke, thrombosis, heparin-induced thrombocytopenia (HIT), Alzheimer's disease, non-alcoholic steatohepatitis, insulin resistance, and/or inflammation

### BRIEF DESCRIPTION OF THE DRAWINGS

FIG. 1 depicts the micro evaporative dissolution analysis of all the Compound 1 spray dry dispersion (SDD) prototypes in pH 6.8 phosphate buffer.

FIG. 2 depicts a comparison of only the top spray dry dispersion (SDD) prototypes in relation to Compound 1 alone.

FIG. 3 depicts mouse PK using a suspension dose in 0.5% methyl cellulose of Compound 1 alone (triangle) or as an SDD at 30 mg/kg (diamond) or 100 mg/kg (square).

### DEFINITIONS

“Therapeutically effective amount,” as used herein, refers broadly to an amount of a compound disclosed herein, that is effective for preventing, ameliorating, treating or delaying the onset of a disease or condition.

“Prophylactically effective amount,” as used herein, refers to an amount of a compound disclosed herein, that is effective for inhibiting the onset or progression of a disorder.

### DETAILED DESCRIPTION OF THE INVENTION

The disclosure is not limited to the particular embodiments described below, as variations of the particular embodiments may be made and still fall within the scope of the appended claims. The terminology employed is for the purpose of describing particular embodiments and is not intended to be limiting.

### 4-((2-hydroxy-3-methoxybenzyl)amino)-benzenesulfonamide Derivative 12-lipoxygenase Inhibitors

Described herein are oral formulations comprising 12-LOX inhibitors.

The pharmaceutical formulations described herein comprise a selective 12-lipoxygenase (12-LOX) inhibitor. In one embodiment, the selective 12-LOX inhibitor is a 4-((2-hydroxy-3-methoxybenzyl)amino)-benzenesulfonamide derivative. In a specific embodiment, the 4-((2-hydroxy-3-methoxybenzyl)amino)-benzenesulfonamide derivative is a compound of Formula (I):

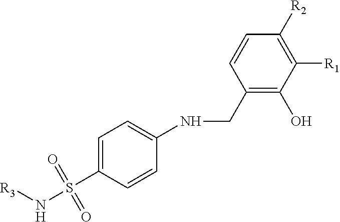

wherein R₁ is methoxy; R₂ is H; and R₃ is selected from the group consisting of 2-benzothiazole, 4-methyl-2-benzothiazole, 6-fluoro-2-benzothiazole, 6-methoxy-2-benzothiazole, 2-benzoxazole, 2-benzimidazole, 2-thiophene, 4-methyl-2-thiazole, 5-methyl-2-thiazole, 4,5-methyl-2-thiazole, 4-phenyl-2-thiazole, 3-quinoline, 8-isoquinoline, phenyl, 1,4-biphenyl, 1-naphthalene, 2-naphthalene, 3-piperazine-phenyl, 4-piperidine-phenyl, 3-piperidine-phenyl, 2-pyridine, 3-pyridine, 4-piperazine-3-pyridine, 3-tert-butyl-phenyl, 3-morpholine-phenyl, 4N-boc-piperidine-3-phenyl, and 3-isopropyl-phenyl.

In an embodiment, the pharmaceutical formulation comprises Compound 1:

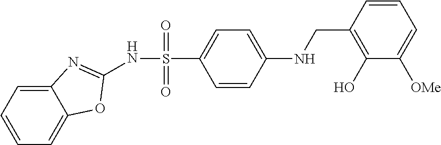 [SMILES: COc1cccc(CNc2ccc(S(=O)(=O)Nc3nc4ccccc4o3)cc2)c1O]

or a pharmaceutically acceptable salt thereof.

In an embodiment, the pharmaceutical formulation comprises Compound 2:

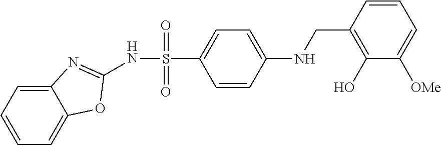 [SMILES: COc1cccc(CNc2ccc(S(=O)(=O)Nc3nc4ccccc4o3)cc2)c1O]

or a pharmaceutically acceptable salt thereof.

In an embodiment, the pharmaceutical formulation comprises Compound 3:

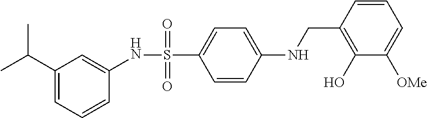 [SMILES: COc1cccc(CNc2ccc(S(=O)(=O)Nc3cccc(C(C)C)c3)cc2)c1O]

or a pharmaceutically acceptable salt thereof.

The pharmaceutical formulations described herein comprise the compounds or salt of Formula (I), or a combination thereof.

The present disclosure is further directed to a pharmaceutical formulation comprising an pharmaceutically acceptable carrier and at least one selective 12-LOX inhibitor compound described herein. In a preferred embodiment, the carrier is a polymer, preferably vinylpyrollidone-vinyl acetate copolymers (Kollidon VA64), amino methacrylate copolymer (Eudragit EPO) or Hydroxypropylmethylcellulose E3 (HPMC E3).

Compounds 1, 2, and 3 were previously identified as selective 12-LOX inhibitors in e.g., U.S. Pat. No. 10,266,488:

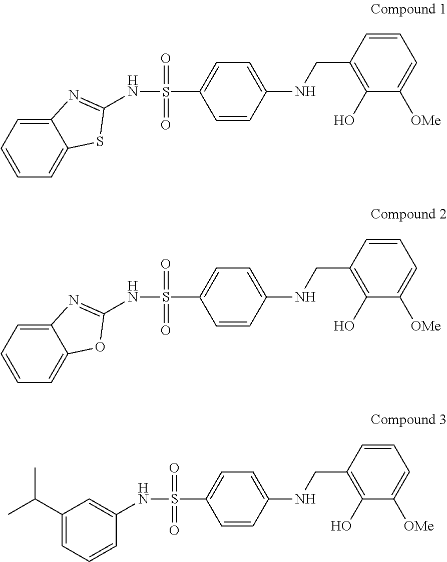 [SMILES: COc1cccc(CNc2ccc(S(=O)(=O)Nc3nc4ccccc4s3)cc2)c1O ; COc1cccc(CNc2ccc(S(=O)(=O)Nc3nc4ccccc4o3)cc2)c1O ; COc1cccc(CNc2ccc(S(=O)(=O)Nc3cccc(C(C)C)c3)cc2)c1O]

These compounds have low solubility in water. Below is a comparison of oral bioavailability of Compound 1 Suspension (Table 1) versus Solution (Table 2).

TABLE 1
Bioavailability in rats from suspension dosing is < 0.5%

| | | Compound 1 Dosage | Tₘₐₓ | T_{1/2} | Cₘₐₓ | AUCₗₐₛₜ | AUC_{0-∞} |
|---|---|---|---|---|---|---|---|
| Route | Group | (mg/kg) | (hr) | (hr) | (ng/mL) | (hr*ng/mL) | (hr*ng/ml) |
| IV | 1 | 3 | 0.083 | 2.58 | 49600 | 43600 | 43600 |
| | 2 | 10 | 0.083 | 2.98 | 91200 | 100000 | 100000 |
| | 3 | 30 | 0.083 | 2.56 | 180000 | 213000 | 214000 |
| Oral | 4 | 3 | 4.00 | 1.82 | 30.0 | 203 | 237 |
| | 5 | 10 | 2.67 | 3.41 | 71.3 | 399 | 478 |
| | 6 | 30 | 2.67 | 2.46 | 142 | 737 | 768 |

TABLE 2
Bioavailability in rats from solution dosing is only 3 %

| | | Dosing | | | | C₀ or | | | |
|---|---|---|---|---|---|---|---|---|---|
| Group | | Level | Cl_obs | T_{1/2} | Tₘₐₓ | Cₘₐₓ | AUCₗₐₛₜ | Vₛₛ_obs | F |
| (N) | Route | (mg/Kg) | (mL/min/Kg) | (hr) | (hr) | (ng/mL) | (hr*ng/ml) | (L/kg) | (%) |
| 3 | IV | 3 | 0.986 | 2.59 | NA | 63725 | 50699 | 0.101 | NA |
| 3 | PO | 30 | NA | 2.69 | 0.25 | 6610 | 15231 | NA | 3.01 |
| | | | | (0.09) | | (3637) | (6808) | | (1.34) |

AUCₗₐₛₜ = area under the time concentration curve from t = 0 to the last observed plasma
concentration timepoint
Cₘₐₓ = maximal plasma concentration
IV = intravenous
PO = peroral
C₀ = plasma concentration extrapolated to T = Oh
Cl_obs = observed clearance
Cₘₐₓ = maximal plasma concentration
F = fraction absorbed
NA = not applicable
T_{1/2} = half-life
Tₘₐₓ = time of maximal plasma concentration

The intravenous formulation uses cyclodextrin (CD) to increase solubility. However, a drug loading of <10% is not suitable for oral formulation. The inventors explored a new approach to oral formulation without guidance from the art including lipid solubility screening in various non-aqueous vehicles, self micro-emulsifying systems with oils, surfactants, and co-surfactants, amorphous solid dispersions via hot melt and spray drying, micronization, and in silico screen of supercritical fluid nanoparticle engineering. Minimal improvement in aqueous solubility and dissolution rate was observed in all cases except for non-aqueous vehicles and spray dried dispersion. While non-aqueous vehicles could provide improved solubility, rapid precipitation in vivo limited oral bioavailability in animals as seen in Table 2. The inventors found that spray dried dispersion (SDD) was the most promising technique showing aqueous solubility enhancement of several orders of magnitude and acceptable oral bioavailability in rodent and dog models. The pharmaceutical acceptable carrier can be a polymer.

### Formulations

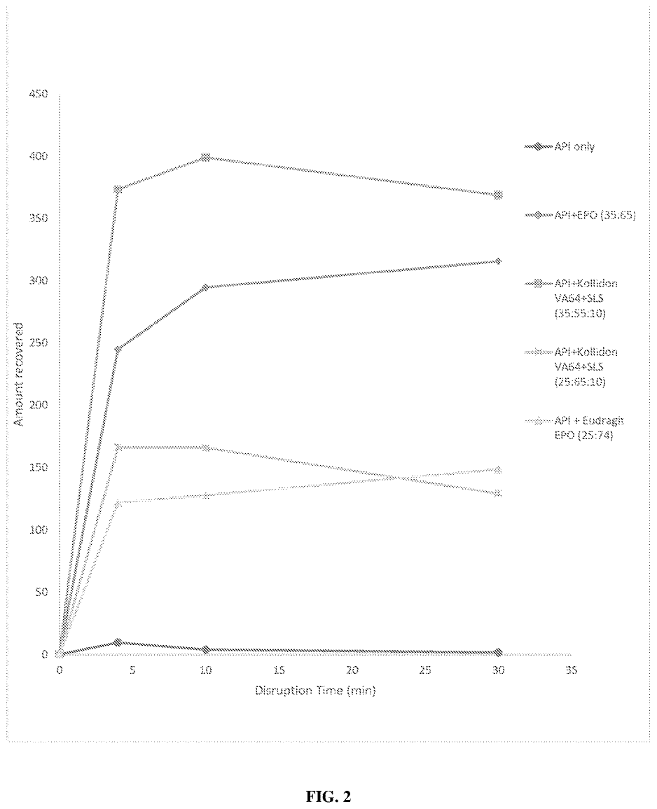

Surprisingly, the inventors found that the majority of solvents utilized in the prior art did not solubilize Compound 1 to an adequate level for an efficient spray drying process or polymer:drug loading. THF was the most suitable solvent, and could be combined with other solvents such as acetone, ethanol and water to enable dissolution of the drug, polymer and additives in the formulation. Furthermore, given the anticipated dose of the product, maximizing the drug loading in the amorphous dispersion was essential. The inventors found that Kollidon VA64, Eudragit EPO and HPMC E3 allowed drug loading of 35% or more in the amorphous dispersion. Surprisingly, higher levels of drug loading in Kollidon VA64, and Eudragit EPO resulted in improved in vitro dissolution results when comparing 25% to 35% drug loading (FIG. 2). HPMC E3 allowed drug loading up to 40% and the in vitro dissolution remained about the same in this polymer matrix from 20 to 40% drug loading.

The formulation comprises a compound described herein and a weight/weight solids composition of 35% Compound 1:55% Kollidon VA64:10% Sodium Lauryl Sulfate (SLS), 35% Compound 1:65% Eudragit EPO or 40% Compound 1:60% HMPC E3.

The inventors found that higher drug loading improved the SDD performance (FIG. 2). The inventors found that the SDD formulation showed good animal bioavailability, about 14%-37% in rodents and about 50% in dog. The SDD formulation showed a 28 to 100-fold increase in bioavailability versus a suspension.

The pharmaceutical acceptable carrier can be a polymer. The pharmaceutical formulations described herein can comprise a polymer selected from the group consisting of vinylpyrollidone-vinyl acetate copolymers (Kollidon VA64), amino methacrylate copolymer (Eudragit EPO), Hydroxypropylmethylcellulose E3 (HPMC E3), or a combination thereof.

The pharmaceutical formulations described herein can comprise a polymer in an amount of between about 40% and 80% of the total solids content. The pharmaceutical formulations described herein can comprise a polymer in an amount of between about 40% and 60%, 50% and 80%, 40% and 50%, or 60% and 70% of the total solids content. The pharmaceutical formulations described herein can comprise a polymer in an amount of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% of the total solids content.

The pharmaceutical formulations described herein can vinylpyrollidone-vinyl acetate copolymers (Kollidon VA64) in an amount of about 40 to 80% of the total solids content.

The pharmaceutical formulation may further contain other acceptable carriers or additives in an amount that does not alter the nature of the formulation, including, vegetable oils such as peanut oil, cotton seed oil, sesame oil, as well as organic solvents, glycerol, and surfactants.

In one embodiment, the pharmaceutical formulation comprises at least 30% drug, preferably 35% or more of drug in the final amorphous polymer matrix.

The compounds described herein can be present in any suitable amount within the pharmaceutical formulations described herein. Those of skill in the art can readily determine suitable concentrations of compound to include in the pharmaceutical formulations depending on various factors including dosage and route of administration. The pharmaceutical formulations useful in the present invention can contain a quantity of a compound described herein in an amount effective to treat or prevent the condition, disorder or disease of the subject being treated.

The compounds described herein, e.g., compounds of Formula (I), optionally Compound 1, Compound 2, Compound 3, or mixtures thereof, can be present in the pharmaceutical formulation in an amount of at least 5%, 10%, 15%, 25%, 30%, 35%, 40%, 45%, 50%, 55%, or 60% of the composition of the final amorphous spray dried dispersion.

The compounds described herein can be present in the pharmaceutical formulation in an amount of about 1 mg to 1,000 mg. The compounds described herein can be present in the pharmaceutical formulation in an amount of about 1 mg to 100 mg; 250 mg to 500 mg, or 250 mg to 750 mg. The compounds described herein can be present in the pharmaceutical formulation in an amount of about 5 mg, 10 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg 275 mg, 280 mg, 285 mg, 290 mg, 295 mg, 300 mg, 205 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 510 mg, 515 mg, 520 mg, 525 mg, 530 mg. 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 625 mg, 655 mg, 675 mg, 700 mg, 725 mg, 750 mg, 775 mg, 800 mg, 825 mg, 850 mg, 875 mg, 900 mg, 925 mg. 950 mg, 975 mg, or 1,000 mg.

The inventors surprisingly discovered that the solubility of the Compounds 1, is greatly enhanced and maintained for a period of time ranging from 10 to 120 minutes in phosphate buffer or simulated intestinal fluid. Because solubility is maintained after the initial dissolution event, the compound has time to be absorbed from the GI tract.

The amorphous spray dried dispersion is manufactured using standard spray drying techniques known to one skilled in the art. The 35:55:10 Compound 1:Kollidon VA 64:SLS is manufactured by making a solvent solution containing THF, methanol and water in 90:5:5 ratio. To the solvent solution compound 1, Kollidon VA64 and SLS are added in a 35:55:10 ratio at a total solids content of 2.65%. The solids are mixed until dissolved and then spray dried using a Buchi B-290. The solids are collected and characterized analytically. Similar techniques known to those skilled in the art are utilized to manufacture the Eudragit EPO and HPMC E3 formulations.

The 35:55:10 Compound 1:Kollidon VA 64:SLS SDD was dosed up to 700 mg/kg, the maximum feasible dose (MFD) due to suspension viscosity and dose volume limitations. No saturation in Cmax or AUC observed up to MFD with SDD dosing

The formulations described herein showed solid stability. For example, after seven months at RT (room temperature (about 25° C.) storage the formulation showed no change in amorphous state. At >18 months RT storage still no change in amorphous state. This stability over a time of a year a and a half was unexpected. Further, the formulation showed dosing suspension stability. A 3 mg/mL suspension was prepared and analyzed for various quality attributes. After 3 days there was no change in Related substances or Crystallinity by XRD.

In addition to the compounds disclosed herein, the pharmaceutical formulations may further comprise at least one of any suitable auxiliaries including, but not limited to, diluents, crystal inhibitors, tonicifiers, water structure forming agents or disruptors, polymers, ion pairing agents, stabilizers, buffers, salts, lipophilic solvents, preservatives, adjuvants or the like. Pharmaceutically acceptable auxiliaries are preferred. Examples and methods of preparing such sterile solutions are well known in the art and can be found in well-known texts such as, but not limited to, REMINGTON'S PHARMACEUTICAL SCIENCES (Adejare, Ed., 23rd Edition, Academic Press. (2020); Handbook of Pharmaceutical Excipients, 9^{th} Edition, Pharmaceutical Press (2020)).

Pharmaceutically acceptable carriers can be routinely selected that are suitable for the mode of administration, solubility and/or stability of the compound.

Pharmaceutical excipients and additives useful in the pharmaceutical formulations described herein can also include, but are not limited to, proteins, peptides, amino acids, lipids, and carbohydrates (e.g., sugars, including monosaccharides, di-, tri-, tetra-, and oligosaccharides; derivatized sugars such as alditols, aldonic acids, esterified sugars; and polysaccharides or sugar polymers), which can be present singly or in combination, comprising alone or in combination in ranges of 1-99.99% by weight or volume. Exemplary protein excipients include serum albumin such as human serum albumin (HSA), recombinant human albumin (rHA), gelatin, and casein. Representative amino acid components, which can also function in a buffering capacity, include alanine, glycine, arginine, betaine, histidine, glutamic acid, aspartic acid, cysteine, lysine, leucine, isoleucine, valine, methionine, phenylalanine, and aspartame.

The pharmaceutical formulations described herein can also include additional therapeutic agents such as argatroban, but not limited other therapeutic agents or combinations thereof.

An additional therapeutic agent can be an additional 12-LOX inhibitor. A 12-LOX inhibitor can be an organic compound, an inorganic compound, a biological compound (e.g., proteins or fragments thereof, antibodies or fragments thereof, nucleic acids, nucleic acid analogs, saccharides, or peptides), or any combination thereof. A 12-LOX inhibitor can also be synthetic or naturally occurring. Selective 12-LOX inhibitors are described in U.S. Pat. Nos. 10,266,488 and 10,752,581.

The mixing ratios of the 12-LOX inhibitors can be optimized to provide maximum therapeutic effects.

Additional agents include but not limited to anti-thrombotic agents such as argatroban, fondaparinux, lepirudin, bivalirudin, danaparoid and drotrecogin alfa; antidiabetic agents such as exenatide, albiglutide, pramlintide, semaglutide, lixisenatide, and dulaglutide. Combinations with direct-acting oral coagulants is also contemplated, including but not limited to apixaban, dabigatran, rivaroxaban, and edoxaban.

The formulations can be presented in unit-dose or multi-dose containers as a powder-in-capsule, powder-in-bottle, stick-pack or powder-in-sachet unit. The formulations can be combined with other external excipients and compressed into a tablet or filled into an empty capsule shell to produce a dosage unit. Solutions and suspensions suitable for oral administration can be prepared from the spray dried powders, granules and tablets. The spray dried powders may be sprinkled on food and administered with a meal.

Methods of preparing pharmaceutical formulations with a certain amount of active ingredients are known, or will be apparent in light of this disclosure, to those skilled in the art. Methods of preparing said pharmaceutical formulations can incorporate other suitable pharmaceutical excipients and their formulations as described in REMINGTON'S PHARMACEUTICAL SCIENCES, above.

Methods of preparing the pharmaceutical preparations described herein are manufactured in a manner that is known, including conventional mixing, dissolving, and spray drying processes.

### Administration and Dosage

One of ordinary skill in the art will appreciate that administration of pharmaceutically effective amounts of the pharmaceutical formulations described herein to a patient in need thereof, can be determined empirically, or by standards currently recognized in the medical arts. It will be understood that, when administered to a human patient, the total daily usage of the agents of the compositions described herein will be decided within the scope of sound medical judgment by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. It is well within the skill of the art to start doses of the agents at levels lower than those required to achieve the desired therapeutic effect and to gradually increase the dosages until the desired effect is achieved.

Routes of administration and dosages of effective amounts of the pharmaceutical formulations comprising the compounds are also disclosed. The preferred route of administration is oral. The pharmaceutical formulations described herein can be administered by a unit dosage as a powder-in-capsule, powder-in-bottle, stick-pack or powder-in-sachet unit. The formulations can be combined with other external excipients and compressed into a tablet or filled into an empty capsule shell to produce a dosage unit. The compounds described herein can be administered in combination with other pharmaceutical agents in a variety of protocols for effective treatment of disease.

The pharmaceutical formulations can be administered in a single daily dose, or the total daily dosage can be administered in divided doses of two, three, or four times daily. Doses maybe administered for one week, one month, or over the course of several months, 3, 6, 9 or 12 months, or intervals known in the art and determined to be clinically relevant. For example, the pharmaceutical formulations described herein can be administered for 7 days, 14 days, 21 days, 28 days, or 35 days. The daily dosage of the formulations can be varied over a wide range from about 1 to about 1,000 mg per patient, per day, more particularly from about 200 to about 700, preferably about 600 mg/day. The range of doses as determined by a practitioner can be adjusted to achieve a plasma and/or serum concentration that is clinically relevant.

Specifically, the pharmaceutical formulations described herein can be administered at least once a day over the course of several weeks, several months, or several years. In one embodiment, the pharmaceutical formulations are administered at least once a day over several weeks to several months. In an embodiment, the pharmaceutical formulations are administered once a day over at least one year or longer.

In one embodiment, the pharmaceutical formulations described herein can be administered orally. The oral dosage can comprise between about 1 and 1,000 mg of the 12-LOX inhibitor in the pharmaceutical formulation. The oral dosage of the 12-LOX inhibitor described herein can be between about 10 and 100 mg, 250 mg and 500 mg, 750 mg and 1,000 mg, or 500 mg and 750 mg. The oral dosage of the 12-LOX inhibitor described herein can be about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 mg.

The pharmaceutical formulations described herein can be administered to any animal that can experience the beneficial effects of the compounds of the invention. Preferred is administration to humans.

The dosage administered will be dependent upon the age, health, and weight of the recipient, kind of concurrent treatment, if any, frequency of treatment, and the nature of the effect desired.

### Therapeutic and Prophylactic Methods 12-LOX Mediated Diseases/Disorders

The disclosure further provides a method for treating or preventing a 12-LOX mediated disease or disorder comprising administering to a mammal a therapeutically or prophylactically effective amount of a pharmaceutical formulation described herein.

The 12-LOX mediated disease or disorder to be treated or prevented is typically a disease or disorder wherein the production of 12-hydroxyeicosatetraenoic acid (12-HETE) is implicated in the development or progression of the disease or disorder. 12-LOX mediated diseases and disorders includes those where 12-LOX is a direct mediator of the diseases and disorders, as well as those where the inhibition of 12-LOX results in therapeutic value in the treatment or prevention of the diseases and disorders.

A method for treating or preventing a 12-LOX mediated disease or disorder can comprise administering to a mammal a therapeutically or prophylactically effective amount of any of compounds of Formula (I), or a salt thereof in the pharmaceutical formulation. In one embodiment, the compound in the formulation is Compound 1.

In one embodiment, the 12-LOX is human 12-LOX.

The 12-LOX mediated disease or disorder can be selected from the group consisting of type 1 diabetes, type 2 diabetes, diabetic nephropathy, diabetic neuropathy, diabetic retinopathy, lupus, cardiovascular disease, Alzheimer's disease, Non-Alcoholic steatohepatitis, platelet hemostasis, skin diseases, heparin-induced thrombocytopenia, thrombosis, and cancer.

The cancer can be selected from the group consisting of prostate cancer, colorectal cancer, breast cancer, and lung cancer. In an embodiment, the cancer is a hematologic cancer.

The cardiovascular disease can be selected from the group consisting of congestive heart failure, myocardial infarction and stroke. The cardiovascular disease can be a mixture of congestive heart failure, myocardial infarction, and stroke

A method of treating or preventing type 1 and/or type 2 diabetes can comprise administering to a mammal a therapeutically or prophylactically effective amount of any of compounds described herein, e.g., compounds of Formula (I), Compound 1, Compound 2, Compound 3, or mixtures thereof, or a salt thereof in the pharmaceutical formulation.

A method of treating or preventing thrombosis can comprise orally administering to a mammal thereof a pharmaceutical formulation comprising a therapeutically or prophylactically effective amount of any of the compounds described herein, a salt thereof.

A method for reducing PAR1-AP or PAR4-AP induced platelet aggregation can comprise orally administering to a mammal thereof a pharmaceutical formulation described herein comprising a therapeutically or prophylactically effective amount of any of compounds described herein, e.g., compounds of Formula (I), Compound 1, Compound 2, Compound 3, or a mixture thereof, a salt thereof.

A method for reducing PAR4-AP induced calcium mobilization can comprise orally administering to a mammal thereof a pharmaceutical formulation comprising a therapeutically or prophylactically effective amount of any of compounds described herein, a salt thereof.

A method for treating U46619-induced and FcγRIIa-mediated platelet aggregation can comprise orally administering to a mammal thereof a pharmaceutical formulation comprising a therapeutically or prophylactically effective amount of any of compounds described herein, a salt thereof.

12-LOX inhibitors can be used in transplantation/xenotransplantation methods, for example, where islets are treated ex vivo to improve survival prior to transplant.

Provided is a method for treating or preventing a disease or disorder in which a FcγRIIa-mediated pathway is involved comprising orally administering a pharmaceutical formulation described herein. Such a disease or disorder can be an immune-mediated thrombocytopenia and thrombosis disorder. Examples of immune-mediated thrombocytopenia and thrombosis disorders include, but are not limited to, heparin-induced thrombocytopenia (HIT); anti-phospholipid syndrome; sepsis syndrome; thrombosis associated with therapeutic or diagnostic monoclonal antibodies; and thrombotic thrombocytopenic purpura. In some aspects, disclosed herein are methods for inhibiting platelet activation and preventing or treating thrombosis.

A method of inhibiting or decreasing platelet activation can comprise contacting a platelet with a pharmaceutical formulation comprising an effective amount of a compound described herein. In some embodiments, the platelet activation is immune-mediated. In some embodiments, the immune-mediated platelet activation is resulting from the activation of a FcγRIIa receptor.

Methods for treating or preventing a thrombotic event, myocardial infarction, or stroke can comprise orally administering to a subject in need thereof a pharmaceutical formulation comprising an effective amount of a compound described herein are also disclosed. Immune-mediated platelet activation can lead to thrombi formation, which can clot arteries and result in stroke, myocardial infarction, organ infarction, limb gangrene, or other serious complications.

The subject can be one who exhibits one or more risk factors for an immune-mediated thrombocytopenia and thrombosis disorder. The subject can be receiving or had recently received heparin therapy. The subject can be undergoing orthopedic surgery. It is known in the art that orthopedic surgery patients are at higher risk for developing HIT than are patients who receive heparin for other medical reasons.

A method for treating or preventing a 12-LOX mediated disease and/or disorder may administer the pharmaceutical formulation comprising Compound 1, Compound 2, Compound 3, or a combination thereof to a subject in need thereof. The subject in need thereof can be a mammal. In a preferred embodiment, the subject in need thereof is a human.

The 12-LOX mediated disease and/or disorder can be type 1 diabetes, type 2 diabetes, diabetic nephropathy, diabetic neuropathy, diabetic neuropathy, diabetic retinopathy, lupus, cardiovascular disease, Alzheimer's disease, Non-Alcoholic steatohepatitis, platelet hemostasis, skin diseases, heparin-induced thrombocytopenia, thrombosis, or cancer.

A method for treating a patient suffering from immune-mediated thrombocytopenia can comprise administering the pharmaceutical formulation comprising Compound 1, Compound 2, Compound 3, or a combination thereof, to a subject in need thereof.

A method for treating a patient suffering from a thrombosis disorder comprising orally administering a pharmaceutical formulation comprising Compound 1, Compound 2, Compound 3, or a combination thereof to a subject in need thereof.

### Methods of Formulation

The compounds described herein, e.g., compounds of Formula (I), for example Compound 1, Compound 2, and Compound 3, show poor solubility in water.

Although preferred embodiments have been depicted and described in detail herein, it will be apparent to those skilled in the relevant art that various modifications, additions, substitutions, can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

The following examples illustrate some embodiments and aspects of the invention. It will be apparent to those skilled in the relevant art that various modifications, additions, substitutions, can be performed without altering the spirit or scope of the invention, and such modifications and variations are encompassed within the scope of the invention as defined in the claims which follow. The following examples do not in any way limit the invention.

### EXAMPLES

### Example 1

### Development of an Oral Formulation for Compound 1

Prior, all oral dosing with Compound 1 had been done with 100% PEG300 as the vehicle. While this vehicle is sufficient for short term efficacy studies, the inventors needed a more robust and clinically relevant formulation for the longer dosing regimens required for mouse efficacy models. Toward the end, the inventors tested doses of Compound 1 as a suspension in 0.5% methylcellulose however the oral bioavailability in both mice and rats was <5%. These results necessitated alternative formulation studies such as an amorphous spray dried dispersion (SDD) in which Compound 1 is formulated with different polymers to improve the dissolution and oral bioavailability.

The inventors used an approach including a screen for solvents, develop an analytical method, screen for polymer matrices and additives, evaluate aqueous dissolution and stability, optimize drug load and yield, and rodent pharmacokinetic (PK) studies.

Solubility in organic solvents relevant to spray draying were conducted (Table 1), showing that THF with EtOH in water was preferred (85/10/5).

TABLE 3
Solvents Tested

| | | Solubility |
|---|---|---|
| Solvent System | Soluble | Concentration |
| 50:50 Acetone:Methanol | No | <2.5 mg/mL |
| 85:10:5 Tetrahydrofuran:Ethanol:Water | Yes | 10 mg/mL |
| 50:45:5 Tetrahydrofuran:Acetone:Water | Yes | 10 mg/mL |
| 95:5 Tetrahydrofuran:Water | Yes | 10 mg/mL |
| 50:50 Methylene Chloride:Methanol | No | <2.5 mg/mL |

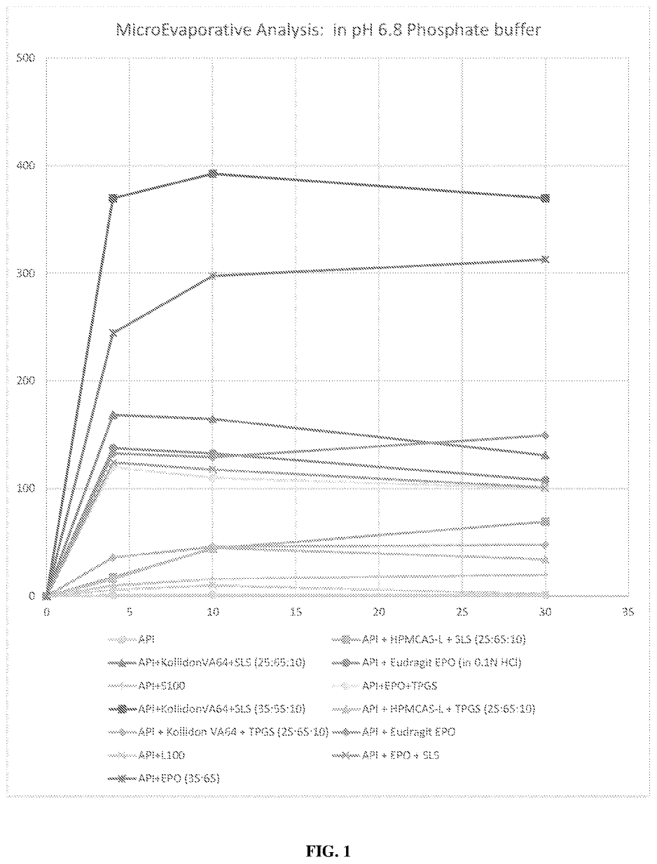

Next, a number of different polymer systems were screened at varying drug loads and surfactants to see which provided the best dissolution. These studies showed that a significant improvement of Compound 1 dissolution was observed with Compound 1:KollidonVA64:SLS (35:55:10). Compared to Compound 1 alone in FIG. 1 and FIG. 2.

The Compound 1:KollidonVA64:SLS (35:55:10) SDD was scaled-up to ~65 g which was enough for the PK and efficacy studies outlined below. Stability studies demonstrated no change after 18 months at room temperature storage. Moreover, as a 3 mg/mL solution, no change in related substances or crystallinity by XRD was observed after 3 days.

### Example 2

### PK Parameters and Dose-Linearity in Mice

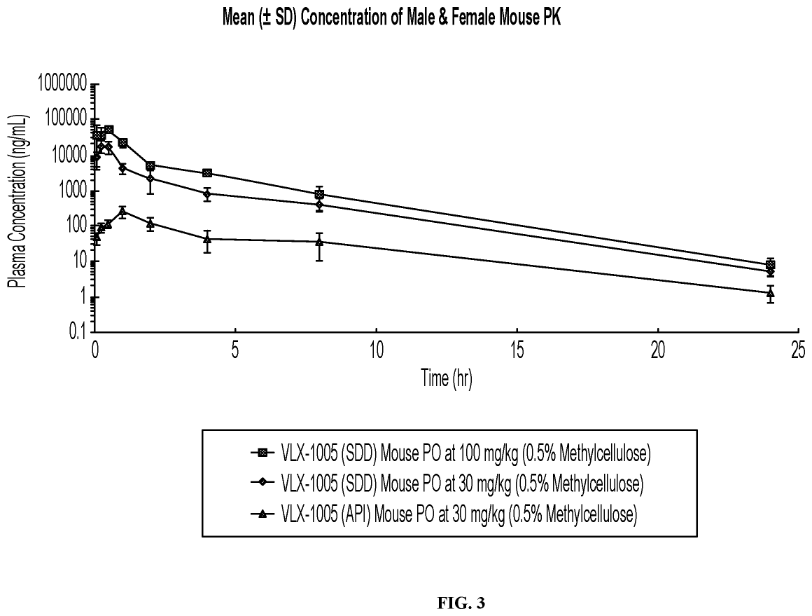

The inventors identified a SDD formulation with significantly improved dissolution in vitro (FIGS. 1 and 2), they confirmed that this improvement was observed in vivo (FIG. 3). The inventors conducted mouse and rat PK using the formulation Compound 1:KollidonVA64:SLS (35:55:10). The oral exposure obtained in mice was significantly improved as shown in FIG. 3 and Table 4.

TABLE 4
PK Parameters for Compound 1:Kollidon VA64:SLS (35:55:10) dosed as a suspension
at both 30 and 100 mg/kg. Mean PK parameters following administration of Compound 1
PO (Spray-Dried Dispersion) in Mice.

| | | Dosing | | | | C₀ or | | | |
|---|---|---|---|---|---|---|---|---|---|
| Group | | Level | Cl_obs | T_{1/2} | Tₘₐₓ | Cₘₐₓ | AUCₗₐₛₜ | Vₛₛ_obs | F |
| (N) | Route | (mg/Kg) | (mL/min/Kg) | (hr) | (hr) | (ng/ml) | (hr*ng/mL) | (L/kg) | (%) |
| 6 | PO (SDD) | 30 | NA | 2.7 | 0.3 | 192954 | 23845 | NA | 16 |
| 4 | PO (SDD) | 100 | NA | 2.3 | 0.4 | 60675 | 71548 | NA | 14 |

AUCₗₐₛₜ = area under the time concentration curve from t = 0 to the last observed plasma
concentration timepoint
Cₘₐₓ = maximal plasma concentration
IV = intravenous
PO = peroral
C₀ = plasma concentration extrapolated to T = Oh
Cl_obs = observed clearance
Cₘₐₓ = maximal plasma concentration
F = fraction absorbed
NA = not applicable
T_{1/2} = half-life
Tₘₐₓ = time of maximal plasma concentration

### Example 3

### Preparation of Spray Dried Dispersion

A Buchi B-290 spray dryer was used to produce the spray dried prototype for Compound 1. The formulation for spray the dried dispersion is reported in Table 5.

TABLE 5
Formulation Table-Spray Drying

| | | | Weight |
|---|---|---|---|
| | % w/w | % w/w | Dispensed |
| Material | (Solids) | (Solution) | (g) |
| Compound 1 | 35.0 | 0.9275 | 13.0 |
| Kollidon VA64 | 55.0 | 1.4575 | 20.43 |
| Sodium Dodecyl Sulfate | 10.0 | 0.2650 | 3.71 |
| (SLS) | | | |
| Tetrahydrofuran (THF) | N/A | 88.2 | 1235 |
| Methanol (MeOH) | N/A | 4.64 | 65 |
| Water (H2O) | N/A | 4.64 | 65 |

The target quantity of THF MeOH and water was dispensed into appropriately sized container and a mixer was engaged to create a vortex. The target quantity of Compound 1 was added into the container. The solution was mixed until all API was dissolved. The API solution was passed through a 200 mesh screen. Target quantities of Kollidon VA64 and SLS were added into the container. The solution was mixed until all the polymer and SLS dissolved. The solution was spray dried using the following parameters in 6.

TABLE 6
Settings Used During Spray Drying

| Parameter | Setting |
|---|---|
| Inlet Temperature (° C.) | 80 |
| Outlet Temperature (° C.) | 40 |
| Aspirator (%) | 80 |
| Condenser Temperature (° C.) | −20 |

The spray dried dispersion collected from the spray dryer was dried (secondary drying) using a conventional heat oven at 40° C. for 12 hours. Samples were collected before and after drying for residual solvent (THF, MeOH) content. Dried material was characterized for assay, impurities, p-XRD and DSC.

Collectively, these data demonstrate that the inventors successfully designed a clinically relevant formulation of Compound 1 that could be utilized in the efficacy studies and for therapeutic purposes. The formulation is not only stable, well-tolerated but also provides excellent exposure of Compound 1 in mice as shown in Table 4. In addition to looking at PK in mice, the inventors tested the Compound 1. Compound formulation in rats and dogs since those are the relevant specific for GLP toxicity studies. These efforts revealed excellent and reproducible exposure in both rats and dogs (% F of 20-50%). The inventors were surprisingly able to dose up to 700 mg/kg in rats.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A pharmaceutical formulation comprising: 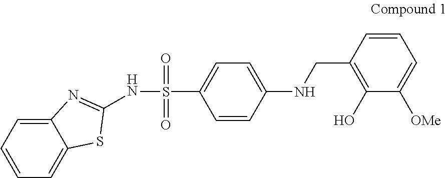 [SMILES: COc1cccc(CNc2ccc(S(=O)(=O)Nc3nc4ccccc4s3)cc2)c1O]
or a pharmaceutically acceptable salt thereof; and a pharmaceutically acceptable carrier.

2. A pharmaceutical formulation comprising: 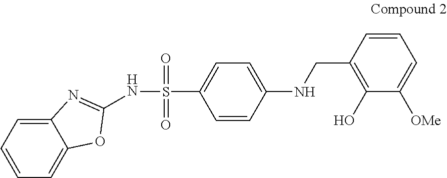 [SMILES: COc1cccc(CNc2ccc(S(=O)(=O)Nc3nc4ccccc4o3)cc2)c1O]
or a pharmaceutically acceptable salt thereof, or diastereomers thereof; and a pharmaceutically acceptable carrier.

3. A pharmaceutical formulation comprising: 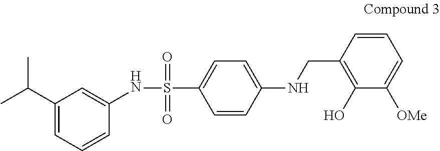 [SMILES: COc1cccc(CNc2ccc(S(=O)(=O)Nc3cccc(C(C)C)c3)cc2)c1O]
or a pharmaceutically acceptable salt thereof, or diastereomers thereof; and a pharmaceutically acceptable carrier.

4. A pharmaceutical formulation comprising a compound of Formula (I): 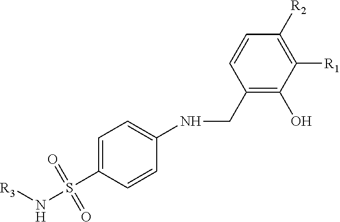
wherein
R₁ is methoxy;
R₂ is H; and
R₃ is selected from the group consisting of 2-benzothiazole, 4-methyl-2-benzothiazole, 6-fluoro-2-benzothiazole, 6-methoxy-2-benzothiazole, 2-benzoxazole, 2-benzimidazole, 2-thiophene, 4-methyl-2-thiazole, 5-methyl-2-thiazole, 4,5-methyl-2-thiazole, 4-phenyl-2-thiazole, 3-quinoline, 8-isoquinoline, phenyl, 1,4-biphenyl, 1-naphthalene, 2-naphthalene, 3-piperazine-phenyl, 4-piperidine-phenyl, 3-piperidine-phenyl, 2-pyridine, 3-pyridine, 4-piperazine-3-pyridine, 3-tert-butyl-phenyl, 3-morpholine-phenyl, 4N-boc-piperidine-3-phenyl, and 3-isopropyl-phenyl, or a pharmaceutically acceptable salt thereof, or diastereomers thereof; and
a pharmaceutically acceptable carrier.

5. The pharmaceutical formulation of any one of claims 1-4, wherein the pharmaceutical acceptable carrier is a polymer.

6. The pharmaceutical formulation of any one of claims 1-5, wherein the pharmaceutical acceptable carrier is a polymer selected from the group consisting of vinylpyrollidone-vinyl acetate copolymers (Kollidon VA64), amino methacrylate copolymer (Eudragit EPO), Hydroxypropylmethylcellulose E3 (HPMC E3), or a combination thereof.

7. The pharmaceutical formulation of any one of claims 1-6, wherein the pharmaceutical acceptable carrier is a polymer in an amount of between about 40% and 80% of the total solids content.

8. The pharmaceutical formulation of any one of claims 1-7, wherein the pharmaceutical acceptable carrier is a polymer in an amount of between about 40% and 60%, 50% and 80%, 40% and 50%, or 60% and 70% of the total solids content.

9. The pharmaceutical formulation of any one of claims 1-8, wherein the pharmaceutical acceptable carrier is a polymer in an amount of about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% of the total solids content.

10. The pharmaceutical formulation of any one of claims 1-9, wherein the polymer is vinylpyrollidone-vinyl acetate copolymers (Kollidon VA64) in an amount of about 40 to 80% of the total solids content.

11. The pharmaceutical formulation of any one of claims 1-10, wherein the compound of Formula (I) is in an amount between about 1 mg to about 1,000 mg.

12. The pharmaceutical formulation of any one of claims 1-11, wherein the compound of Formula (I) is in an amount between about 1 mg to 100 mg; 250 mg to 500 mg, or 250 mg to 750 mg.

13. The pharmaceutical formulation of any one of claims 1-12, wherein the compound of Formula (I) is in an amount of about 5 mg, 10 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg 275 mg, 280 mg, 285 mg, 290 mg, 295 mg, 300 mg, 205 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 625 mg, 655 mg, 675 mg, 700 mg, 725 mg, 750 mg, 775 mg, 800 mg, 825 mg, 850 mg, 875 mg, 900 mg, 925 mg, 950 mg, 975 mg, or 1,000 mg.

14. The pharmaceutical formulation of any one of claims 1-13, wherein Compound 1, Compound 2, Compound 3, or a combination thereof is in an amount between about 1 mg to about 1,000 mg.

15. The pharmaceutical formulation of any one of claims 1-14, wherein Compound 1, Compound 2, Compound 3, or a combination thereof is in an amount between about 1 mg to 100 mg; 250 mg to 500 mg, or 250 mg to 750 mg.

16. The pharmaceutical formulation of any one of claims 1-15, wherein Compound 1, Compound 2, Compound 3, or a combination thereof is in an amount of about 5 mg, 10 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg 275 mg, 280 mg, 285 mg, 290 mg, 295 mg, 300 mg, 205 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 625 mg, 655 mg, 675 mg, 700 mg, 725 mg, 750 mg, 775 mg, 800 mg, 825 mg, 850 mg, 875 mg, 900 mg, 925 mg, 950 mg, 975 mg, or 1,000 mg.

17. The pharmaceutical formulation of any one of claims 1-16, wherein the formulation further comprises a pharmaceutically acceptable carrier, additive, excipient, preservative, solvent, buffer, or a mixture thereof.

18. The pharmaceutical formulation of any one of claims 1-17, wherein the formulation is suitable for oral administration.

19. The pharmaceutical formulation of any one of claims 1-18 formulated for use as a therapeutic agent in the treatment or prevention of 12-LOX mediated diseases and disorders.

20. A pharmaceutical composition for treating or preventing a 12-LOX mediated disease and/or disorder comprising the pharmaceutical formulation of any one of claims 1-19.

21. A pharmaceutical composition for reducing PAR1-AP or PAR4-AP induced platelet aggregation comprising the pharmaceutical formulation of any one of claims 1-19.

22. A pharmaceutical composition for reducing PAR4-AP induced calcium mobilization comprising the pharmaceutical formulation of any one of claims 1-19.

23. A pharmaceutical composition for reducing PAR4-AP induced calcium mobilization comprising the pharmaceutical formulation of any one of claims 1-19.

24. The pharmaceutical compositions of any one of claims 20-23, wherein the pharmaceutical formulation is formulated for oral administration.

25. A method for treating or preventing a 12-LOX mediated disease and/or disorder comprising administering the pharmaceutical formulation of any one of claims 1-19 to a mammal in need thereof.

26. A method for reducing PAR1-AP or PAR4-AP induced platelet aggregation comprising orally administering to a mammal thereof the pharmaceutical formulation of any one of claims 1-19.

27. A method for reducing PAR4-AP induced calcium mobilization comprising orally administering to a mammal thereof the pharmaceutical formulation of any one of claims 1-19.

28. A method for treating U46619-induced and FcγRIIa-mediated platelet aggregation comprising orally administering to a mammal thereof the pharmaceutical formulation of any one of claims 1-18.

29. The method of any one of claims 25-28, wherein the mammal is a human.

30. The method of any one of claims 25-29, wherein the subject in need thereof has a 12-LOX mediated disease and/or disorder.

31. The method of any one of claims 25-30, wherein the 12-LOX mediated disease and/or disorder is selected from the group consisting of: as skin diseases and platelet hemostasis, transplantation/xenotransplantation, cancer, optionally prostate cancer, colorectal cancer, breast cancer and lung cancer, type 1 and type 2 diabetes, diabetic nephropathy, optionally diabetic nephropathy, diabetic retinopathy, lupus, cardiovascular disease, optionally myocardial infarction, congestive heart failure, heart failure, and stroke, thrombosis, heparin-induced thrombocytopenia (HIT), Alzheimer's disease, non-alcoholic steatohepatitis, insulin resistance, and inflammation.
